(19) United States
(12) Patent Application Publication
(10) Pub. No.: US 2003/0220377 A1
(43) Pub. Date: Nov. 27, 2003

(54) Indole compounds and their use as estrogen agonists/antagonists

(76) Inventor: Richard Chesworth, Mystic, CT (US)

Correspondence Address: PFIZER INC., PATENT DEPARTMENT, MS8260-1611, EASTERN POINT ROAD, GROTON, CT, 06340, US

(21) Appl. No.: 10/431,282

(22) Filed: May 7, 2003

Related U.S. Application Data
(60) Provisional application No. 60/379,161, filed on May 8, 2002.

(51) Int. Cl. A61K 31/5377; A61K 31/4245; A61K 31/425; A61K 31/42; A61K 31/404; C07D 413/02; C 07D 4 3/02

(52) U.S. Cl. 514/362; 514/378; 514/372; 514/414; 514/233.5; 544/143; 548/131; 548/240; 548/465; 548/206

(57) ABSTRACT

This invention relates to compounds, in particular indoles, that are useful as estrogen agonists and antagonists and pharmaceutical uses thereof. The present invention also relates to indoles that are selective for the ERβ receptor and pharmaceutical uses thereof. The compounds have utility in that they may be used to treat estrogen mediated disorders.

### FIELD OF THE INVENTION

This invention relates to compounds, in particular indoles, that are useful as estrogen agonists and antagonists and pharmaceutical uses thereof. The present invention also relates to indoles that are selective for the ERβ receptor and pharmaceutical uses thereof.

### BACKGROUND OF THE INVENTION

As a mediator of the actions of estrogenic hormones, the estrogen receptor (ER) plays a central role in regulating a diverse array of normal physiological processes involved in the development and function of the reproductive system, as well as many other aspects of health, such as bone density, cardiovascular health, etc.

It is known that compounds that bind to the ER are potentially useful in the treatment of a wide range of disease states. These include estrogen agonists for treatment of disease linked to estrogen deficiency, such as osteoporosis, cardiovascular and neurodegenerative diseases in post menopausal women; and estrogen antagonists for treatment of breast and uterine cancer. Furthermore, it is known that certain ligands, such as tamoxifen display mixed agonist/antagonist action; that is they are either estrogen agonists, estrogen antagonists or a partial estrogen antagonist when binding to the estrogen receptors of different tissues.

Estrogen is the agent of choice in preventing osteoporosis or post menopausal bone loss in women, it is the only treatment that unequivocally reduces fractures. However, estrogen stimulates the uterus and is associated with an increased risk of endometrial cancer. Although the risk of endometrial cancer is thought to be reduced by concurrent use of a progestogen, there remains concern about possible increased risk of breast cancer with the use of estrogen.

It would be desirable to be able to produce ligands which are recognizable by and able to bind to the estrogen receptor. Further, it would be desirable to produce ligands having estrogen-like function, but which are devoid of unwanted side-effects of estrogenic compounds. For example, osteoporosis is greatly ameliorated by the use of fully active estrogens; however, due to the recognized risk of uterine cancer in patients treated chronically with active estrogens, it is not clinically advisable to treat osteoporosis with fully active estrogens for prolonged periods.

Until recently, it has been assumed that estrogen binds to a single estrogen receptor (ER) in cells, causing conformational changes that result in release from heat shock proteins and binding of the receptor as a dimer to the so-called estrogen response element in the promoter region of a variety of genes.

Recently, a second estrogen receptor, ERβ, has been identified and cloned (Katzenellenbogen and Korach Endocrinology 138, 861-2 (1997). ERβ, and the classical ER, renamed ERα, have significantly different amino acid sequences in the ligand binding domain and carboxy-terminal transactivation domains ( approximately 56% amino acid identity) and only 20% homology in their amino-terminal transactivation domain. This suggests that some ligands may have higher affinity to one receptor over the other. Further, ligand-dependent conformational changes of the two receptors, and interaction with co-factors, will result in very different biological actions of a single ligand. In other words, a ligand that acts as an agonist on ERα may very well serve as an antagonist on ERβ. An example of such behavior has been described by Paech et al. (Science 277, 1508-1510, 1997). In addition, it has been found that there are differences in the proportion of expression of ERβ and ERα in different organs. For example, organs in which there is a high proportion of ERα receptors include the uterus and the hypothalmus. ERβ is highly in the ovaries and the bone.

With the recent identification of ERβ, and the recognition that ERβ and ERα have different tissue distribution, ER-selective modulators would possess significant clinical utility. Further, ER-selective modulators that have the capacity to selectively bind or to activate the ER subtypes, ERβ and ERα would be useful in elucidating the biology of the two receptors and might assist in the development of estrogen pharmaceuticals with improved tissue selectivity.

### SUMMARY OF THE INVENTION

In a first aspect, the invention relates to a compound of formula (I)

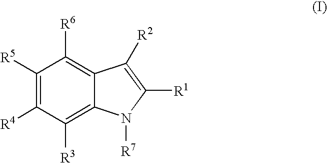

or the pharmaceutically acceptable salts thereof; wherein:

R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl

wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:

halogen; (C₁-C₆)alkyl; (C₃-C₈)cycloalkyl; (C₄-C₈)cycloalkenyl; (C₁-C₆)alkoxy; hydroxy; R¹² CO₂, R¹²R¹³NCO, R¹²R¹³N; (C₁-C₆) alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆)alkyl; R¹²R¹³N(C₁-C₆)alkoxy; R¹²R¹³N(C₁-C₆alkyl)S; N-morpholino(CH₂)ₙO; or R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:

halogen, (C₁-C₆)alkyl; (C₃-C₈)cycloalkyl; (C₄-C₈)cycloalkenyl; (C₁-C₆)alkoxy; hydroxy; R¹²CO₂; R¹²R¹³NCO; R¹²R¹³N;(C₁-C₆)alkylcarbonyl; —CHO; cyano; thio; R¹² SO₂(C₁-C₆)alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆) alkoxy; R¹²CO(C₁-C₆)alkoxy; R¹²R¹³N SO₂(C₁-C₆)alkyl; and R¹²R¹³N SO₂(C₁-C₆) alkoxy

wherein:

R¹² and R¹³ are each independently selected from the group consisting of hydrogen; halogen; (C₁-C₇)alkyl; (C₃-C₈)cycloalkyl; (C₄-C₈)cycloalkenyl; (C₆-C₁₀) aryl; (C₂-C₁₀)alkenyl, (C₂-C₁₀)alkynyl; (C₂-C₄)heteroaryl; (C₁-C₆)alkylaryl; (C₁-C₆) alkyl(C₂-C₆)heteroaryl; (C₂-C₆)alkoxyaryl; (C₂-C₆)alkoxy(C₂-C₆)heteroaryl; or R¹² and R¹³ taken together form a three to eight membered heterocyclic ring having 1 to 3 heteroatoms; n is from 0 to 5; and x is 1 or 2;

or R¹ and R² are each independently a group of the formula:

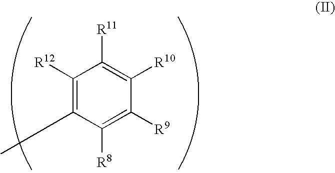

wherein R⁸, R⁹, R¹¹ and R¹²are each independently hydrogen; hydroxy; (C₁-C₆) alkyl; (C₁-C₆)alkoxy; or halogen;

R¹⁰ is hydrogen; hydroxy; (C₁-C₆)alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy;

R³, R⁴, R⁵and R⁶ are each independently hydrogen, hydroxy; (C₁-C₆)alkyl; (C₁-C₆)alkoxy; or halogen; and

R⁷ is H or (C₁-C₃)alkyl;

with the proviso that at least one of R¹ or R² must be the group of formula (II) and with the proviso that when R¹ and R² are each independently the group of Formula II, wherein each R¹⁰ is hydrogen or hydroxy, then at least one of R³, R⁴, R⁵or R⁶ must be other than hydrogen, hydroxy or (C₁-C₆)alkoxy.

Before the present compositions and methods are disclosed and described, it is to be understood that this invention is not limited to specific systemic methods or to particular formulations, as such may, of course, vary. It is also to be understood that the terminology used herein is for the purpose of describing particular embodiments only, and is not intended to be limiting.

Throughout the application, where publications (including, but not limited to, U.S. Patents) are referenced, the disclosures of these publications in their entireties are hereby incorporated by reference into this application in order to more fully describe the state of the art to which this invention pertains.

In the specification and claims that follow, reference will be made to a number of terms which shall be defined to have the following meaning.

The singular forms “a”, “an” and “the” include plural referents unless the context clearly dictates otherwise.

“Optional” or “optionally” means that the subsequently described event or circumstance may or may not occur, and that the description includes instances where the event or circumstances occurs and instances where it does not.

The term “alkyl” refers to straight or branched, monovalent, saturated aliphatic chains having the designated number of carbon atoms and includes, but is not limited to methyl, ethyl, propyl, isopropyl, butyl, isobutyl, t-butyl, pentyl, isopentyl, and hexyl.

The term “alkenyl” refers to straight or branched chain hydrocarbon groups of 2 to 10 carbon atoms having at least one double bond.

The term “alkynyl” refers to straight of branched chain hydrocarbon groups of 2 to 10 carbon atoms having at least one triple bond.

The term “aryl” refers to monocylic and polycyclic aromatic groups, or fused ring systems having at least one aromatic ring, having from 3 to 14 backbone atoms. Examples of aryl groups include, without limitation, phenyl, naphthyl, dihydronaphthyl, tetrahydronapthyl, and the like.

“Cycloalkyl” groups include means a cyclic hydrocarbon. Examples of cycloalkyl groups include cyclopropyl, cyclobutyl, cyclopentyl, cyclohexyl and cycloheptyl. Preferred cycloalkyl groups are (C₃-C₈)cycloalkyl. It is also possible for the cycloalkyl group to have one or more double bonds, but is not aromatic. Cycloalkyl having at least one double bond are herein referred to as “cycloalkenyl” groups. Examples of cycloalkyl groups having at least one double bond include cyclopentenyl, cyclohexenyl, cyclohexadienyl, cyclobutadienyl, and the like.

“Heteroaryl” means an aromatic ring containing one or more heteroatoms. If the heteroaryl group contains more than one heteroatom, the heteroatom may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, benzo[b]thienyl, isooxazolyl, isothiazolyl and thiodiazolyl.

The term “heteroatom” includes oxygen, nitrogen and sulphur.

The term “substituted” means that a hydrogen atom on a molecule has been replaced with a different atom or molecule. The atom or molecule replacing the atom is denoted as a “substituent.” The term “substituted” specifically envisions and allows for substitutions that are common in the art. However, it is generally understood by those skilled in the art that the substituents should be selected so as to not adversely affect the pharmacological characteristics or adversely interfere with the use of the medicament. Suitable substituents include halogen; (C₁-C₆)alkyl; (C₃-C₈)cycloalkyl; (C₄-C₈)cycloalkenyl; (C₁-C₆)alkoxy; hydroxy; R¹²CO₂; R¹²R¹³NCO; R¹²R¹³N; (C₁-C₆) alkylcarbonyl; CHO; cyano; thio; (C₁-C₆)alkylthio; (C₁-C₆)alkylsulfonyl; (C₁-C₆)alkylsulfinyl; CH₂OH; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)O; R¹²R¹³N(C₁-C₆)S; N-morpholino(CH₂)ₙO; and —R¹²R¹³N(CH₂)S(O)ₓ. R¹² and R¹³ are as defined in Formula (I).

When the term “alkyl” is used to suffix another group, such as in “arylalkyl”, “heterocycloalkyl”, “cycloalkylalkyl,” or “heteroarylalkyl” the term defines with more specificity at least one of the groups that a substituted alkyl will contain. In other words, in these instances the specifically named groups are bonded directly through a substituted or unsubstituted alkyl chain, as defined.

An “estrogen agonist/antagonist” is a compound that affects some of the same receptors that estrogen does, but not all, and in some instances, it antagonizes or blocks estrogen. Estrogen agonists/antagonists may also be referred to as antiestrogens although they have some estrogenic activity at some estrogen receptors.

The term “prodrug” refers to compounds that are drug precursors which, following administration, release the drug in vivo via some chemical or physiological process (e.g. a prodrug on being brought to the physiological pH or through enzyme action is converted to the desired drug form).

The term “Estrogen Receptor” as used herein refers to ERβ and/or the ERα. “Estrogen Receptor Modulators” are compounds that bind to the ERβ and/or the ERα receptors and function as estrogen agonists/estrogen antagonists.

An “ERβ selective estrogen receptor modulator” is a compound that selectively binds to the ERβ receptor. By “selective” it is meant that the compound exhibits at least 5 times the binding affinity for the ERβ than the ERα receptor as indicated by IC₅₀ in a competitive binding assay. By “more selective” it is meant that the compound exhibits at least 20 times the binding affinity for the ERβ than the ERα receptor as indicated by IC₅₀ in a competitive binding assay. It is preferable that the compounds of the present invention have an IC₅₀ with respect to ERβ and/or ERα of no more than 500 nanomolar.

By “selectively antagonizing or agonizing” as used in the present specification, it is meant that the compound is selective or more selective for the ERβ receptor and exhibits agonist and/or antagonist activity.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The phrase “pharmaceutically acceptable” indicates that the substance or composition must be compatible chemically and/or toxicologically, with the other ingredients comprising a formulation, and/or the mammal being treated therewith.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. Where more than one basic moiety exists the expression includes multiple salts (e.g., di-salt). The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

The term “female sexual dysfunction” as used herein includes hypoactive sexual desire disorder, sexual anhedonia and dyspareunia. Hypoactive sexual desire disorder is a disorder in which sexual fantasies and desire for sexual activity are persistently or recurrently diminished or absent, causing marked distress of interpersonal difficulties. Hypoactive sexual desire disorder may be lifelong or acquired, generalized (global) or situational (partner-specific). Sexual desire is a complex psychosomatic process based on brain activity (the “generator” or “motor” running in a rheostatic cyclic fashion), a poorly defined hormonal milieu, and cognitive scripting that includes sexual aspiration and motivation. Desynchronization of these components results in hypoactive sexual desire disorder.

Sexual anhedonia (decreased or absent pleasure in sexual activity) is not an official diagnosis. It is almost always classified under hypoactive sexual desire disorder, because loss of pleasure almost always results in loss of desire (although loss of desire may occur first). The cause is likely to be depression or drugs if anhedonia is acquired and global (with all partners in all situations); interpersonal factors if anhedonia is confined to one partner or one situation; or repressive factors (eg. guilt, shame) due to family dysfunction or childhood trauma if anhedonia is lifelong. Sexual aversion is the probable diagnosis in lifelong cases.

Dyspareunia is painful coitus or attempted coitus. Dyspareunia is usually introital but may also occur before, during, or after intercourse. Causes include menopausal involution with dryness and thinning of the mucosa. Pain during or after coitus is the chief complaint.

A chemist of ordinary skill will recognize that certain compounds of this invention will contain atoms which may be in a particular optical or geometric configuration, including but not limited to stereoisomers, diastereomers and mixtures thereof. All such isomers are included in this invention in reference to compounds (I) Similarly, a chemist of ordinary skill will recognize that various pharmaceutically acceptable esters and salts may be prepared from certain compounds of this invention. All such esters and salts are included in this invention in reference to compounds (I).

The present invention relates to compounds that have activity as estrogen receptor modulators, as well as pharmaceutical compositions containing one or more of such compounds and methods of use related to the same. As estrogen receptor modulators, the compounds of this invention have utility in the treatment of a wide range of estrogen-related conditions. In this context, the term “treatment” includes both treatment and/or prevention of an estrogen-related condition. Thus, the compounds of this invention may be administered as a therapeutic and/or prophylactic agent. Certain compounds within the class of estrogen receptor modulators as described herein were found to be selective for the ERβ receptor, and certain compounds within the class of ERβ selective compounds were found to be more selective for the ERβ receptor.

The compounds of the invention are useful as they may act as estrogen receptor modulators in mammalian tissue. It is known that certain diseases are mediated by the estrogen receptor.

Diseases or disorders that are mediated by the estrogen receptor include, but are not limited to, perimenopausal or postmenopausal syndrome, osteoporosis, atrophy of skin or vagina, elevated serum cholesterol levels, cardiovascular disease, Alzheimer's disease, estrogen dependent cancers, including breast or uterine cancer, a prostatic disease, benign prostatic hyperplasia, prostate cancer, obesity, endometriosis, bone loss, uterine fibrosis, aortal smooth muscle cell proliferation, lack of birth control, female sexual dysfunction, acne, hirsutism, dysfunctional uterine bleeding, dysmenorrehea, male infertility, impotence, psychological and behavioral symptoms during menstruation, ulcerative mucositis, uterine fibroid disease, restenosis, atherosclerosis, musculoaponeurotic fibromatosis, alopecia, auto immune disease, cartilage degeneration, delayed puberty, demyelinating disease, dysmyelinating disease, hypoglycemia, lupus erythematosus, myocardial infection, ischemia, thromboembolic disorder, obsessive compulsive disorder, ovarian dysgenesis, post menopausal central nervous system (CNS) disorder, pulmonary hypertension, reperfusion damage, resistant neoplasm, rheumatoid arthritis, seborrhea, sexual precocity, thyroiditis, Turner's syndrome, and hyperlipidemia and female sexual dysfunction. The compounds of the present invention are also useful for blocking a calcium channel, inhibiting an environmental estrogen, minimizing the uterotropic effect of tamoxifen or its analogs, removing fibrin by inhibiting plasminogen activators, inhibiting estrogen positive primary tumors of the brain and CNS, increasing sphincter competence, increasing libido, inhibiting fertility, oxidizing low density lipoprotein, increasing macrophage function, expressing thrombomodulin, or increasing levels of endogenous growth hormone.

When referenced in the present specification, “effective amounts” of the compounds of the invention generally include any amount sufficient to detectably modulate estrogen receptor activity by the assays described herein, by other activity assays known to those of ordinary skill in the art, or by detecting prevention or alleviation of symptoms in a subject afflicted with an estrogen receptor-mediated disorder.

In the methods of this invention as described in the present invention, conditions which present with low bone mass include such conditions as, for example, osteoporosis, childhood idiopathic bone loss, alveolar bone loss, mandibular bone loss, bone fracture, osteotomy, bone loss associated with periodontitis and prosthetic ingrowth. In a further embodiment, the condition which presents with low bone mass is osteoporosis.

In one embodiment of the first aspect of the invention the following compounds are excluded from formula (I):

4-(2-Phenyl-1H-indol-3-yl)-phenol; 2-(4-Hydroxy-phenyl)-3-phenyl-1H-indol-6-ol; 3-(4-Hydroxy-phenyl)-2-phenyl-1H-indol-6-ol; 2,3-Bis-(4-hydroxy-phenyl)-1H-indole; 2,3-Bis-(4-hydroxy-phenyl)-1H-indol-6-ol; 2,3-Diphenyl-1H-indol-5-ol; 2,3-Diphenyl-1H-indol-6-ol; 4-(1-Methyl-2-phenyl-1H-indol-3-yl)-phenol; 4-(1-Ethyl-2-phenyl-1H-indol-3-yl)-phenol; 1-Ethyl-2,3-diphenyl-1H-indol-6-ol; 1-Ethyl-2-(4-hydroxy-phenyl)-3-phenyl-1H-indol-6-ol; 1-Ethyl-3-(4-hydroxy-phenyl)-2-phenyl-1H-indol-6-ol; 4-(3-Phenyl-1H-indol-2-yl)-phenol; [2-(2,3-Diphenyl-1H-indol-6-yloxy)-ethyl]-diethyl-amine; Diethyl-{2-[4-(3-phenyl-1H-indol-2-yl)-phenoxy]-ethyl}-amine; 2-(4-Methoxy-phenyl)-3-phenyl-1H-indol-6-ol and 2-(4-Methoxy-phenyl)-3-phenyl-6-(2-pyrrolidin-1-yl-ethoxy)-1H-indole.

In a further embodiment of the first aspect of the invention, R¹ is phenyl or (C₂-C₆)heteroaryl. In an even further embodiment, the (C₂-C₆) heteroaryl is thienyl; furyl; pyrrolyl; isoxazolyl; isothiazoyl or thiodiazolyl.

In a further embodiment of the first aspect of the invention, R² is a group of formula (II). In a further embodiment R² is a group of formula (II), wherein R⁸, R⁹, R¹ and R¹² are hydrogen and R¹⁰ is hydroxy or (C₁-C₆)alkoxy.

In a further embodiment of the first aspect of the invention R³, R⁴, R⁵ and R⁶ are hydrogen.

In a further embodiment of the first aspect of the invention R¹ is phenyl or (C₂-C₆)heteroaryl; R² is a group of formula (II); and R³, R⁴, R⁵ and R⁶ are hydrogen. In yet another embodiment of the first aspect of the invention, the (C₂-C₆)heteroaryl is thienyl; furyl; pyrrolyl; isoxazolyl; isothiazoyl or thiodiazolyl; R⁸, R⁹, R¹¹ and R¹² are hydrogen; and R¹⁰ is hydroxy or (C₁-C₆)alkoxy.

In a further embodiment of the first aspect of the invention, the compound of Formula (I) is selected from the group consisting of:

2,3-Bis-(4-methoxy-phenyl)-1H-indole;

5-Chloro-2,3-diphenyl-1H-indole;

5-Chloro-2,3-bis-(2-chloro-phenyl)-1H-indole;

4-[2-(4-Methoxy-phenyl)-1H-indol-3-yl]-phenol;

5-Fluoro-2,3-diphenyl-1H-indole;

2-(4-Methoxy-phenyl)-3-phenyl-1H-indole;

2,3-Bis-(4-fluoro-phenyl)-1H-indole;

4-[5-Chloro-3-(4-methoxy-phenyl)-1H-indol-2-yl]-phenol;

2,3-Bis-(4-hydroxy-phenyl)-5-chloro-1H-indole;

2,3-Bis-(4-hydroxy-phenyl)-7-chloro-1H-indole;

2,3-Bis-(4-hydroxy-phenyl)-4-chloro-1H-indole;

2,3-Bis-(4-hydroxy-phenyl)-6-chloro-1H-indole;

4-[5-Bromo-2-(4-methoxy-phenyl)-1H-indol-3-yl]-phenol;

2,3-Bis-(4-hydroxy-phenyl)-6-chloro-1H-indole;

2,3-Diphenyl-1H-indol-4-ol;

4-(7-Chloro-2-phenyl-1H-indol-3-yl)-phenol;

2,3-Bis-(4-hydroxy-phenyl)-1-methyl-indole;

4-(2-Thiophen-2-yl-1H-indol-3-yl)-phenol; and

4-{2-[4-(2-Pyrrolidin-1-yl-ethoxy)-phenyl]-1H-indol-3-yl}-phenol.

4-[2-(1-Methyl-1H-pyrrol-2-yl)-1H-indol-3-yl]-phenol;

4-[2-(3-Methyl-isoxazyl-4-yl)-1H-indol-3-yl]-phenol;

4-[2-(5-Methyl-isoxazyl-4-yl)-1H-indol-3-yl]-phenol;

4-[2-(3,5-Dimethyl-isoxazyl-4-yl)-1H-indol-3-yl]-phenol;

4-[3-(4-Hydroxy-phenyl)-1H-indol-2-yl]-benzoic acid methyl ester;

4-[3-(4-Hydroxy-phenyl)-1H-indol-2-yl]-benzoic acid ethyl ester;

4-[3-(4-Hydroxy-phenyl)-1H-indol-2-yl]-benzoic acid isopropylester;

4-(2-Isothiazol-5-yl-1H-indol-3-yl)-phenol;

4-[2-(4-Methyl-isothiazol-5-yl)-1H-indol-3-yl]-phenol;

4-(2-Cyclopropyl-1H -indol-3-yl)-phenol;

4-[2-(3-Ethyl-isoxazyl-4-yl)-1H-indol-3-yl]-phenol;

4-[2-(5-Methyl-furan-3-yl)-1H-indol-3-yl]-phenol; and

4-(2-Furan-3-yl-1H-indol-3-yl)-phenol.

In a second aspect, the invention relates to a pharmaceutical composition for antagonizing or agonizing the estrogen receptor in a mammal comprising an estrogen receptor antagonizing or agonizing effective amount of a compound of formula (I) or a pharmaceutically accepted salt thereof, and a pharmaceutically acceptable carrier.

In a third aspect, the invention relates to a pharmaceutical composition for selectively antagonizing or agonizing the ERβ estrogen receptor in a mammal comprising administering an ERβ estrogen receptor antagonizing or agonizing effective amount of a compound of formula (I) or the pharmaceutically accepted salts thereof

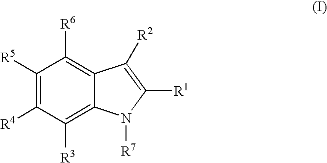

wherein:

R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl;

wherein the (C₁-C₆)alkyl; phenyl; (C₂-C₆)heteroaryl; (C₃-C₈)cycloalkyl; or (C₄-C₈) cycloalkenyl groups of R¹ or R² are optionally substituted by from 1 to 3 substituents independently selected from the group consisting of:

halogen; (C₁-C₆)alkyl; (C₃-C₈)cycloalkyl; (C₄-C₈)cycloalkenyl; (C₁-C₆)alkoxy; hydroxy; R¹² CO², R¹²R¹³NCO, R¹²R¹³N; (C₁-C₆) alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)alkoxy; R¹²R¹³N(C₁-C₆)alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:

halogen, (C₁-C₆)alkyl; (C₃-C₈)cycloalkyl; (C₄-C₈)cycloalkenyl; (C₁-C₆)alkoxy, hydroxy, R¹²CO₂, R¹²R¹³NCO, R¹²R¹³N;(C₁-C₆)alkylcarbonyl, —CHO, cyano, thio; R¹²SO₂(C₁-C₆)alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆)alkoxy; R₁₂CO(C₁-C₆)alkoxy; R¹²R¹³N SO₂(C₁-C₆)alkyl; and R¹²R¹³N SO₂(C₁-C₆) alkoxy

wherein:

R¹² and R¹³ are each independently selected from the group consisting of hydrogen; halogen; (C₁-C₇)alkyl; (C₃-C₈)cycloalkyl; (C₄-C₈)cycloalkenyl; (C₆-C₁₀) aryl; (C₂-C₁₀)alkenyl, (C₂-C₁₀)alkynyl; (C₂-C₄)heteroaryl; (C₁-C₆)alkylaryl; (C₁-C₆)alkyl (C₂-C₆)heteroaryl; (C₂-C₆)alkoxyaryl; (C₂-C₆) alkoxy(C₂-C₆)heteroaryl; or R¹² and R¹³ taken together form a three to eight membered heterocyclic ring having up to 3 heteroatoms; n is from 0 to 5; and x is 1 or 2;

or R¹ and R² are each independently a group of the formula:

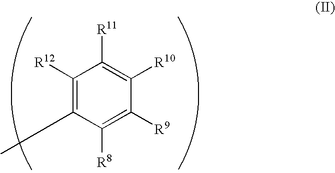

wherein R⁸, R⁹, R¹¹ and R¹² are each independently hydrogen; hydroxy; (C₁-C₆)alkyl; (C₁-C₆)alkoxy; or halogen;

R¹⁰ is hydrogen; hydroxy; (C₁-C₆)alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy;

R³, R⁴, R⁵ and R⁶ are each independently hydrogen, hydroxy; (C₁-C₆)alkyl; (C₁-C₆)alkoxy; or halogen; and

R⁷ is H or (C₁-C₃)alkyl;

with the proviso that at least one of R¹ or R² must be the group of formula (II);

and a pharmaceutically acceptable carrier.

In one embodiment of the third aspect of the invention, the compounds are compounds of Formula I with the proviso that when R¹ and R² are each independently the group of Formula II, wherein each R¹⁰ is hydrogen or hydroxy, then at least one of R³, R⁴, R⁵ or R⁶ must be other than hydrogen, hydroxy or (C₁-C₆)alkoxy.

In one embodiment of the third aspect of the invention R¹ in formula (I) is (C₂-C₆) heteroaryl. In a further embodiment of the further aspect of the invention, the (C₂-C₆) heteroaryl is thienyl; furyl; pyrrolyl; isoxazolyl; isothiazoyl or thiodiazolyl.

In a further embodiment of the third aspect of the invention, R² in formula (I) is a group of formula (II). In a further embodiment, R² in formula (I) is a group of formula (II); R⁸, R⁹, R¹¹ and R¹² are hydrogen and R¹⁰ is hydroxy. In an even embodiment of the third aspect, R² in formula (I) is a group of formula (II); R⁸, R⁹, R¹¹ and R¹² are hydrogen; R¹⁰ is hydroxy and R³, R⁴, R⁵ and R⁶ in formula (I) are hydrogen.

In an even further embodiment of the third aspect of the invention, in the compound of formula (I), R¹ is (C₂-C₆)heteroaryl; R² is a group of formula (II); and R³, R⁴, R⁵ and R⁶ are hydrogen. In an even further embodiment, in the compound of formula (I), the (C₂-C₆)heteroaryl is thienyl; furyl; pyrrolyl; isoxazolyl; isothiazoyl or thiodiazolyl; R⁸, R⁹, R¹¹ and R¹² are hydrogen; and R¹⁰ is hydroxy.

In a fourth aspect, the invention relates to a pharmaceutical composition comprising an agent selected from the group consisting of an anabolic agent; a growth hormone; a growth hormone secretagogue; a prostaglandin agonist/antagonist; a parathyroid hormone; sodium fluoride; and a mixture thereof; the pharmaceutical composition further comprising a compound of formula (I) as set forth in the first aspect of the invention.

In a fifth aspect, the invention relates to a method of treating a condition which presents with low bone mass in a mammal comprising administering to the mammal a compound of formula (I) according to the first aspect of the invention, a prodrug thereof or a pharmaceutically acceptable salt, or a diastereomeric mixture of said compound, salt or prodrug. In a further embodiment of the fifth aspect, the condition is osteoporosis.

In a sixth aspect the invention relates to a kit comprising: a) an amount of a compound of Formula (I) as defined in the first aspect of the invention; b) an amount of a second compound an anabolic agent; a growth hormone; a growth hormone secretagogue; a prostaglandin agonist/antagonist; a parathyroid hormone; sodium fluoride; or a mixture thereof; and c) a container.

In a seventh aspect, the invention relates to a method of treating a disease mediated by the estrogen receptor in a mammal, comprising administering to the mammal a therapeutically effective amount of a compound of formula (I) as set forth in the first aspect of the invention. In one embodiment of this aspect, the disease is selected from the group consisting of perimenopausal or postmenopausal syndrome, osteoporosis, atrophy of skin or vagina, elevated serum cholesterol levels, cardiovascular disease, Alzheimer's disease, estrogen dependent cancers, including breast or uterine cancer, a prostatic disease, benign prostatic hyperplasia, prostate cancer, obesity, endometriosis, bone loss, uterine fibrosis, aortal smooth muscle cell proliferation, lack of birth control, acne, hirsutism, dysfunctional uterine bleeding, dysmenorrehea, male infertility, impotence, psychological and behavioral symptoms during menstruation, ulcerative mucositis, uterine fibroid disease, restenosis, atherosclerosis, musculoaponeurotic fibromatosis, alopecia, auto immune disease, cartilage degeneration, delayed puberty, demyelinating disease, dysmyelinating disease, hypoglycemia, lupus erythematosus, myocardial infection, ischemia, thromboembolic disorder, obsessive compulsive disorder, ovarian dysgenesis, post menopausal CNS disorder, pulmonary hypertension, reperfusion damage, resistant neoplasm, rheumatoid arthritis, seborrhea, sexual precocity, thyroiditis, Turner's syndrome, and hyperlipidemia and female sexual dysfunction.

In an eight aspect, the invention relates to a method for selectively antagonizing or agonizing the ERβ estrogen receptor in a mammal comprising an ERβ estrogen receptor antagonizing or agonizing effective amount of a compound of formula (I) as set forth in the third aspect of the invention, or the pharmaceutically acceptable salts thereof.

Examples of compounds of formula (I) that are effective as estrogen receptor modulators according to the present invention include, but are not limited to

2,3-Bis-(4-methoxy-phenyl)-1H-indole;

5-Chloro-2,3-diphenyl-1H-indole;

5-Chloro-2,3-bis-(2-chloro-phenyl)-1H-indole;

4-[2-(4-Methoxy-phenyl)-1H-indol-3-yl]-phenol;

5-Fluoro-2,3-diphenyl-1H-indole;

2-(4-Methoxy-phenyl)-3-phenyl-1H-indole;

2,3-Bis-(4-fluoro-phenyl)-1H-indole;

4-[5-Chloro-3-(4-methoxy-phenyl)-1H-indol-2-yl]-phenol;

2,3-Bis-(4-hydroxy-phenyl)-5-chloro-1H-indole;

2,3-Bis-(4-hydroxy-phenyl)-7-chloro-1H-indole;

2,3-Bis-(4-hydroxy-phenyl)-4-chloro-1H-indole;

2,3-Bis-(4-hydroxy-phenyl)-6-chloro-1H-indole;

4-[5-Bromo-2-(4-methoxy-phenyl)-1H-indol-3-yl]-phenol;

2,3-Bis-(4-hydroxy-phenyl)-6-chloro-1H-indole;

2,3-Diphenyl-1H-indol-4-ol;

4-(7-Chloro-2-phenyl-1H-indol-3-yl)-phenol;

2,3-Bis-(4-hydroxy-phenyl)-1-methyl-indole;

4-(2-Thiophen-2-yl-1H-indol-3-yl)-phenol; and

4-{2-[4-(2-Pyrrolidin-1-yl-ethoxy)-phenyl]-1H-indol-3-yl}-phenol.

4-[2-(1-Methyl-1H-pyrrol-2-yl)-1H-indol-3-yl]-phenol;

4-[2-(3-Methyl-isoxazyl-4-yl)-1H-indol-3-yl]-phenol;

4-[2-(5-Methyl-isoxazyl-4-yl)-1H-indol-3-yl]-phenol;

4-[2-(3,5-Dimethyl-isoxazyl-4-yl)-1H-indol-3-yl]-phenol;

4-[3-(4-Hydroxy-phenyl)-1H-indol-2-yl]-benzoic acid methyl ester;

4-[3-(4-Hydroxy-phenyl)-1H-indol-2-yl]-benzoic acid ethyl ester;

4-[3-(4-Hydroxy-phenyl)-1H-indol-2-yl]-benzoic acid isopropylester;

4-(2-Isothiazol-5-yl-1H-indol-3-yl)-phenol;

4-[2-(4-Methyl-isothiazol-5-yl)-1H-indol-3-yl]-phenol;

4-(2-Cyclopropyl-1H -indol-3-yl)-phenol;

4-[2-(3-Ethyl-isoxazyl-4-yl)-1H-indol-3-yl]-phenol;

4-[2-(5-Methyl-furan-3-yl)-1H-indol-3-yl]-phenol; and

4-(2-Furan-3-yl-1H-indol-3-yl)-phenol.

As described, in an even further aspect of the invention, it was unexpectedly found that compounds of formula (I) were selective for the ERβ receptor. In order to determine whether a compound is selective or more selective for the ERβ receptor, an assay may be performed as described as described in the present specification in the section entitled “assay for estrogen receptor binding activity”. Compounds that are selective for the ERβ receptor according to the present invention include, but are not limited to:

5-Chloro-2,3-di-o-tolyl-1H-indole;

Diethyl-{2-[4-(3-phenyl-1H-indol-2-yl)-phenoxy]-ethyl}-amine;

2,3-Diphenyl-1H-indole;

5-Chloro-2,3-diphenyl-1H-indole;

5-Chloro-2,3-bis-(2-chloro-phenyl)-1H-indole;

4-[2-(4-Methoxy-phenyl)-1H-indol-3-yl]-phenol;

2,3-Bis-(4-hydroxy-phenyl)-1H-indole;

5-Fluoro-2,3-diphenyl-1H-indole;

2-(4-Methoxy-phenyl)-3-phenyl-1H-indole;

2,3-Bis-(4-fluoro-phenyl)-1H-indole;

4-(3-Phenyl-1H-indol-2-yl)-phenol;

4-[5-Chloro-3-(4-methoxy-phenyl)-1H-indol-2-yl]-phenol;2,3-Bis-(4-hydroxy-phenyl)-5-chloro-1H-indole;

4-(2-Phenyl-1H-indol-3-yl)-phenol;

4-[5-Bromo-2-(4-methoxy-phenyl)-1H-indol-3-yl]-phenol;

2,3-Bis-(4-hydroxy-phenyl)-5-bromo-1H-indole;4-(1-Methyl-2-phenyl-1H-indol-3-yl) -phenol, and

4-(2-Thiophen-2-yl-1H-indol-3-yl)-phenol.

Compounds of the present invention that are more selective for the ERβ receptor include, but are not limited to:

5-Chloro-2,3-diphenyl-1H-indole;

4-[2-(4-Methoxy-phenyl)-1H-indol-3-yl]-phenol;

5-Fluoro-2,3-diphenyl-1H-indole;

4-(3-Phenyl-1H-indol-2-yl)-phenol;

4-[5-Chloro-3-(4-methoxy-phenyl)-1H-indol-2-yl]-phenol;

4-(2-Phenyl-1H-indol-3-yl)-phenol; and

4-[5-Bromo-2-(4-methoxy-phenyl)-1H-indol-3-yl]-phenol.

Compounds that are selective or more selective for the ERβ receptor have the advantage that they could be used in treatments specifically designed to target certain tissue containing ERβ receptors. This would avoid unnecessarily agonizing or antagonizing other receptors in tissue, for example ERα receptors, and would thus avoid potential problems.

The compounds of this invention, as defined in each aspect of the invention, may be administered to mammals (including humans) orally or parenterally in the conventional form of preparations, such as capsules, microcapsules, tablets, granules, powder, troches, pills, suppositories, injections, suspensions and syrups. Suitable formulations may be prepared by methods commonly employed using conventional organic or inorganic additives, such as excipients, binders, disintegrators, lubricants, flavoring agents, stabilizers, dispersing agents, diluents, preservatives, and a base wax. The amount of the active ingredient in the preparation may be at a level that will exercise the desired therapeutic effect. The active ingredient may be usually administered once to four times a day with a unit dosage of 0.1 mg to 50 mg in human patients, but the above dosage may be properly varied depending on the age, body weight and medical condition of the patient and the type of administration.

The compounds of the present invention, as defined in each aspect of the invention, may also be used in combination with other agents to provide sustained therapeutic and prophylactic effects. The compounds of the present invention may be used with other agents including, but not limited to, an anabolic agent; a growth hormone; a growth hormone secretagogue; a prostaglandin agonist/antagonist; a parathyroid hormone; sodium fluoride; or a mixture thereof.

Any prostaglandin agonist/antagonist may be used in combination with the compounds of this invention. The term prostaglandin agonist/antagonist refers to compounds which bind to prostaglandin receptors (e.g., An S. et al., Cloning and Expression of the EP₂ Subtype of Human Receptors for Prostaglandin E₂, Biochemical and Biophysical Research Communications, 1993, 197(1):263-270) and mimic the action of prostaglandin in vivo (e.g., stimulate bone formation and increase bone mass). Such actions are readily determined by those skilled in the art of standard assays. Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296. A variety of these compounds are described and referenced below. However, other prostaglandin agonists/antagonists will be known to those skilled in the art. Exemplary prostaglandin agonists/antagonists are disclosed as follows.

Commonly assigned U.S. Pat. No. 3,932,389, the disclosure of which is incorporated herein by reference, discloses 2-descarboxy-2-(tetrazol-5-yl)-11-desoxy-15-substituted-omega-pentanorprostaglandins useful for bone formation activity.

Commonly assigned U.S. Pat. No. 4,018,892, the disclosure of which is incorporated herein by reference, discloses 16-aryl-13,14-dihydro-PGE₂ p-biphenyl esters useful for bone formation activity.

Commonly assigned U.S. Pat. No. 4,219,483, the disclosure of which is incorporated herein by reference, discloses 2,3,6-substituted-4-pyrones useful for bone formation activity.

Commonly assigned U.S. Pat. No. 4,132,847, the disclosure of which is incorporated herein by reference, discloses 2,3,6-substituted-4-pyrones useful for bone formation activity.

U.S. Pat. No. 4,000,309, the disclosure of which is incorporated herein by reference, discloses 16-aryl-13,14-dihydro-PGE₂ p-biphenyl esters useful for bone formation activity.

U.S. Pat. No. 3,982,016, the disclosure of which is incorporated herein by reference, discloses 16-aryl-13,14-dihydro-PGE₂ p-biphenyl esters useful for bone formation activity.

U.S. Pat. No. 4,621,100, the disclosure of which is incorporated herein by reference, discloses substituted cyclopentanes useful for bone formation activity.

U.S. Pat. No. 5,216,183, the disclosure of which is incorporated herein by reference, discloses cyclopentanones useful for bone formation activity.

Sodium fluoride may be used in combination with the compounds of this invention. The term “sodium fluoride” refers to sodium fluoride in all its forms (e.g., slow release sodium fluoride, sustained release sodium fluoride). Sustained release sodium fluoride is disclosed in U.S. Pat. No. 4,904,478, the disclosure of which is incorporated herein by reference. The activity of sodium fluoride is readily determined by those skilled in the art of biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296).

Any parathyroid hormone (PTH) may be used in combination with the compounds of this invention. The term parathyroid hormone refers to parathyroid hormone, fragments or metabolites thereof and structural analogs thereof which can stimulate bone formation and increase bone mass. Also included are parathyroid hormone related peptides and active fragments and analogs of parathyroid related peptides (see PCT publication no. WO 94/01460). Such bone anabolic functional activity is readily determined by those skilled in the art of standard assays (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below. However, other parathyroid hormones will be known to those skilled in the art. Exemplary parathyroid hormones are disclosed in the following references.

“Human Parathyroid Peptide Treatment of Vertebral Osteoporosis”, Osteoporosis Int., 3, (Supp 1):199-203.

“PTH 1-34 Treatment of Osteoporosis with Added Hormone Replacement Therapy: Biochemical, Kinetic and Histological Responses” Osteoporosis Int. 1:162-170.

Any growth hormone or growth hormone secretagogue may be used in combination with the compounds of this invention. The term “growth hormone secretagogue” refers to a compound which stimulates the release of growth hormone or mimics the action of growth hormone (e.g., increases bone formation leading to increased bone mass). Such actions are readily determined by those skilled in the art of standard assays well known to those of skill in the art. A variety of these compounds are disclosed in the following published PCT patent applications: WO 95/14666; WO 95/13069; WO 94/19367; WO 94/13696; and WO 95/34311. However, other growth hormones or growth hormone secretagogues will be known to those skilled in the art.

In particular a preferred growth hormone secretagogue is N-[1(R)-[1,2-Dihydro-1-methanesulfonylspiro[3H-indole-3,4′-piperidin]-1′-yl)carbonyl]-2-(phenylmethyloxy)ethyl]-2-amino-2-methylpropanamide: MK-677.

Other preferred growth hormone secretagogues include

2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide or its L-tartaric acid salt;

2-amino-N-(1-(R)-benzyloxymethyl-2-(3a-(R)-(4-fluoro-benzyl)-2-methyl-3-oxo-2,3,3a,4,6,7-hexahydro-pyrazolo[4,3-c]pyridin-5-yl)-2-oxo-ethyl)isobutyramide;

2-amino-N-(2-(3a-(R)-benzyl-3-oxo-2,3,3a,4,6,7-hexahydro-pyrazolo[4,3-c]pyridin-5-yl)-1-(R)benzyloxymethyl-2-oxo-ethyl)isobutyramide; and

2-amino-N-(1-(2,4-difluoro-benzyloxymethyl)-2-oxo-2-(3-oxo-3a-pyridin-2-ylmethyl-2-(2,2,2-trifluoro-ethyl)-2,3,3a,4,6,7-hexahydro-pyrazolo[4,3-c]pyridin-5-yl)-ethyl)-2-methyl-propionamide.

It will be recognized that prodrugs and pharmaceutically acceptable salts may be formed from the compounds used as the second compounds in the combinations and kits of the invention. All of such prodrugs and pharmaceutically acceptable salts so formed are within the scope of this invention.

### DETAILED DESCRIPTION OF THE INVENTION

The following reaction schemes illustrate the preparation of the compounds of the present invention. Unless otherwise indicated, the substitutents in the reaction scheme and the discussion that follows are defined as above.

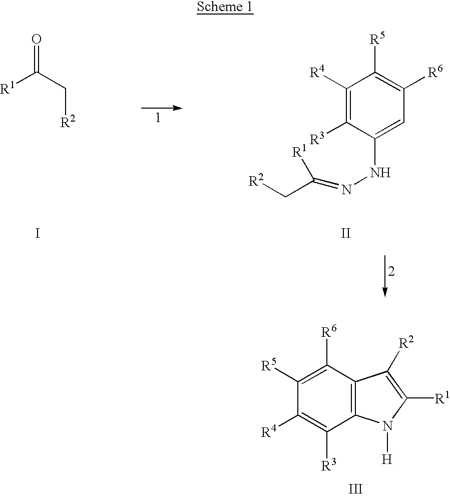

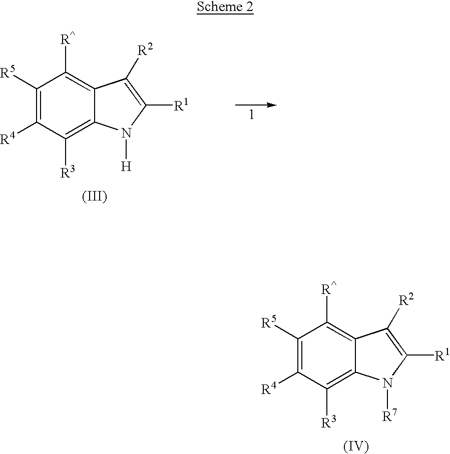

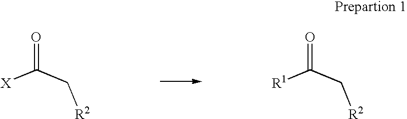

In reaction 1 of scheme 1 the ketone compound of formula (I) is condensed with hydrazine or suitable hydrazine derivatives to form hydrazone (II). The reaction is conducted in the presence of an acid catalyst such as paratoluenesulfonic acid, methanesulfonic acid and hydrochloric acid (HCL), preferably HCl. The reaction is conducted in an inert solvent such as toluene, methylene chloride, tetrahydrofuran (THF) or mixtures thereof. The reaction mixture is stirred at a temperature of from about room temperature to about 110° C., preferably about 100° C., for a time period of from about 1 to about 48 hours, preferably about 24 hours.

The hydrazone (II) may be reacted in situ or after isolation with an acid, such as hydrochloric acid, polyphosphoric acid, boron trifluoride or phosphorous trichloride, preferably with hydrochloric acid or phosphorous trichloride to affect cyclization to the indole (IV), as shown in reaction 2 of scheme 1. The reaction may be conducted in an inert solvent such as toluene, methylene chloride, THF or mixtures thereof at from room temperature to about 110° C., preferably at about 100° C. The reaction mixture is stirred for a period of time of from about 1 to about 48 hours, preferably about 24 hours.

In the scheme above, if R¹ and/or R² have a hydroxyl substituent, or if R³, R⁴, R⁵ or R⁶ are hydroxyl, it is preferable to protect the hydroxyl substituents through the use of protecting groups for all hydroxyls. Protection may be effected by treatment of the compound containing the hydroxyl substituent with a strong base such as sodium hydride (NaH), sodium hexamethyldisilazide (NaHMDS) or potassium hexamethyidisilazide (KHMDS) and reaction with an electrophile such as an alkyl halide, such as methyl iodide or benzyl bromide. The reaction may take place in an inert solvent, such as diethyl ether, dimethylformamide (DMF), THF, toluene or a mixture thereof at a temperature of from about 10° C. to about 100° C., preferably at about room temperature. Removal of the protecting groups may be effected by treatment with hydrogen in the presence of a metal catalyst such as platinium, nickel or palladium, preferably palladium ( this is for benzyl protecting groups) in an inert solvent such as THF, EtOH or MeOH preferably EtOH at a temperature of room temperature to 100° C., preferably at room temperature. Methyl ether protecting groups can be removed by treatment with boron tribromide in an inert solvent such as methylene chloride or 1,2 dichloroethane, preferably as methylene chloride at a temperature of −78° C. to reflux, preferably at 0° C. Preferred protecting groups are methoxy and benzyl. Greene, T. W.; Wuts, P. G. M. Protective Groups in Organic Synthesis, 2^{nd} edition, John Wiley and Sons, Inc. New York, 1991, incorporated herein by reference in its entirety, provides a general description of protecting groups and their uses.

As set forth in scheme 2, in reaction 1, the substituted indoles (IV) can be prepared by treatment of the indole (III) with a strong base such as NaH, NaHMDS or KHMDS and reaction with an electrophile having an appropriate R⁷ substituent, such as an alkyl halide (such as methyl iodide, where R⁷ is methyl) in an inert solvent such as diethyl ether, DMF, toluene or mixtures thereof, preferably at a temperature of from about 10° C. to about 100° C., preferably about room temperature.

As shown in preparation 1, Ketone compounds (I) can be prepared from phenacetyl derivatives such as acid chlorides or N(Me),OMe amides where R² is phenyl, heteroaryl or substituted phenyl. X may be N(CH₃)OCH₃ and halogen, preferably chlorine. Ketones (I) may be prepared by treatment of the phenacetyl derivatives with a suitable nucleophile, such as an organometallic reagent, such as an organolithium, magnesium or cerium reagent, in an inert solvent such as ethyl ether (Et₂O) or THF at a temperature of from about −78° C. to about 100° C., preferably about −78° C. for a time period of from about 30 minutes to about 24 hours, preferably about 2 hours.

Alternatively, an aromatic ring can act as the nucleophile in the presence of a suitable Lewis acid catalyst such as aluminum trichloride (AlCl₃) tin tetrachloride (SnCl₄) or titanium tetrachloride (TiCl₄) in an inert solvent such as methylene chloride. The reaction may be conducted at a temperature of from about −20° C. to about 100° C., preferably about 0° C. to give ketone compounds (I). The reaction may be conducted at a time period of from for a time period of from about 30 minutes to about 24 hours, preferably about 2 hours.

The subject invention also includes isotopically-labelled compounds, which are identical to those recited in Formulas (I) but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention (including the prodrugs thereof and the pharmaceutically acceptable salts of the compounds and the prodrugs) which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples below, by substituting a readily available isotopically labelled reagent for a non-isotopically labelled reagent.

As mentioned, compounds of the present invention may act as antagonists or agonists. The antagonist/agonist activity of the compounds may be determined by any method known in the art. For example, estrogenic activity in human breast cancer MCF7 cells and primary rat granulosa cells may be assessed by transient transfection of an estrogen responsive ERE3-TK-lux luciferase reporter vector essentially as has been described previously in other cell backgrounds, as in, Petersen D N, Tkalcevic G T, Koza-Taylor P H, Turi T G & Brown T A (1998) Identification of estrogen receptor β2, a functional variant of estrogen receptor expressed in normal rat tissue. Endocrinology 139: 1082-1092, incorporated herein by reference in its entirety. The MCF7 cell activity was considered to be mediated through ERα and the granulosa activity was considered to be mediated through ERβ. MCF7 cells may be obtained from ATCC (Manassas, Va.) and transfected with Lipofectamine Plus (Gibco/BRL, Rocville, Md.) as described by the manufacturers. Luciferase may be measured 24 hours after compound addition. Primary rat granulosa cells may be isolated and transfected with ERE3-TK-lux as described in O'Brien M L, Park K, In Y, & Park-Sarge O-K (1999) Characterization of estrogen receptor-β (ERβ) messenger ribonucleic acid and protein expression in rat granulosa cells. Endocrinology 140: 4530-4541, incorporated herein by reference in its entirety.

The invention has been described in detail with particular reference to specific embodiments thereof, but it will be understood that various modifications can be effected within the scope of the invention.

Other features and advantages will be apparent from this description and claims that describe the invention.

### ASSAY FOR ESTROGEN RECEPTOR BINDING ACTIVITY

cDNA cloning of human ERα and ERβ: The coding region of human ERα was cloned by reverse transcriptase polymerase chain reaction (RT-PCR) from human breast cancer cell mRNA using EXPAND High Fidelity PCR System according to manufacturer's instructions (Boehringer-Mannheim, Indianapolis, Ind.). The coding region of human ERβ was cloned by RT-PCR from human testes and pituitary mRNA using EXPAND High Fidelity PCR System according to manufacturer's instructions (Boehringer-Mannheim, Indianapolis, Ind.). PCR products were cloned into pCR2.1 TA Cloning Kit (Invitrogen, Carlsbad, Calif.) and sequenced. Each receptor coding region was subcloned into the mammalian expression vector pcDNA3 ((Invitrogen, Carlsbad, Calif.).

Mammalian cell expression. Receptor proteins were overexpressed in 293T cells. These cells, derived from HEK293 cells (ATCC, Manassas, Va.), have been engineered to stably express large T antigen and can therefore replicate plasmids containing a SV40 origin of replication to high copy numbers. 293T cells were transfected with either hERα-pcDNA3 or hERβ-pcDNA3 using lipofectamine as described by the manufacturer (Gibco/BRL, Bethesda, Md.). Cells were harvested in phosphate buffered saline (PBS) with 0.5 mM EDTA at 48 h post-transfection. Cell pellets were washed once with PBS/EDTA. Whole cell lysates were prepared by homogenization in TEG buffer (50 mM Tris pH 7.4, 1.5 mM EDTA, 50 mM NaCl, 10% glycerol, 5 mM DTT, 5 μg/ml aprotinin, 10 μg/ml leupeptin, 0.1 mg/ml Pefabloc) using a dounce homogenizor. Extracts were centrifuged at 100,000×g for 2 h at 4C. and supernatants were collected. Total protein concentrations were determined using BioRad reagent (BioRad, Hercules, Calif.).

Competition binding assay. The ability of various compounds to inhibit [³H]-estradiol binding was measured by a competition binding assay using dextran-coated charcoal as has been described (Leake R E, Habib F 1987 Steroid hormone receptors: assay and characterization. In: B. Green and R. E. Leake (eds). Steroid Hormones a Practical Approach. IRL Press Ltd, Oxford. 67-92.) 293T cell extracts expressing either hERα or hERβ were incubated in the presence of increasing concentrations of competitor and a fixed concentration of [³H]-estradiol (141 Ci/mmol, New England Nuclear, Boston, Mass.) in 50 mM TrisHCl pH 7.4, 1.5 mM EDTA, 50 mM NaCl, 10% glycerol, 5 mM DTT, 0.5 mg/mL β-lactoglobulin in a final volume of 0.2 mL. All competitors were dissolved in dimethylsulfoxide. The final concentration of receptor was 50 pM with 0.5 nM [³H]-estradiol. After 16 h at 4C., dextran-coated charcoal (20 μL) was added. After 15 min at room temperature the charcoal was removed by centrifugation and the radioactive ligand present in the supernatant was measured by scintillation counting. All reagents were obtained from Sigma (St. Louis, Mo.) unless otherwise indicated. Binding assay results are IC₅₀ values and are reported in nanomoles (nmol) below each compound in the examples that follow.

General Experimental Procedures

NMR spectra were recorded on a Varian Unity 400 spectrometer (Varian Co., Palo Alto, Calif.) at about 23° C. at 400 MHz for proton nuclei. Chemical shifts are expressed in parts per million. The peak shapes are denoted as follows: s, singlet; d, doublet; t, triplet; q, quartet; m, multiplet; bs, broad singlet. Atmospheric pressure chemical ionization (APCl) mass spectra were obtained on a Fisons Platform II Spectrometer (Micromass Inc., Beverly, Mass.). Where the intensity of chlorine or bromine-containing ions are described the expected intensity ratio was observed (approximately 3:1 for ³⁵Cl/³⁷Cl-containing ions) and 1:1 for ⁷⁹Br/⁸¹Br-containing ions) and the intensity of only the lower mass ion is given.

Medium pressure chromatography was performed using a Biotage purification system (Biotage, Dyax Corporation, Charlottesville, Va.) under nitrogen pressure. Flash chromatography was performed with either Baker Silica Gel (40 μm) (J. T. Baker, Phillipsburg, N.J.) or Silica Gel 60 (EM Sciences, Gibbstown, N.J.) in glass columns under low nitrogen pressure. Radial Chromatography was performed using a Chromatotron (model 7924T, Harrison Research, Palo Alto, Calif.). Preparative Chromatography was performed using Analtech Uniplates Silica Gel GF (20×20 cm) (Analtech, Inc. Newark, Del.). Dimethylformamide (DMF), tetrahydrofuran (THF), and dichloromethane (CH₂Cl₂) used as reaction solvents were the anhydrous grade supplied by Aldrich Chemical Company (Milwaukee, Wis.). The term “concentrated” refers to removal of solvent at water aspirator pressure on a rotary evaporator. The term “EtOAc” means ethyl acetate. The abbreviation ‘h’ stands for hours. The term “TBAF” refers to tetrabutylammonium fluoride. The term “DMAP” refers to dimethylaminopyridine. The terms “dichloromethane” and “methylene chloride” are synonymous and are used interchangeably throughout this description and in the Examples and Preparations.

### EXAMPLES

The following examples are set forth to provide those of ordinary skill in the art with a complete description of how the compositions of matter and methods claimed herein are made and evaluated, and are not intended to limit the scope of what the inventors regard as their invention. The activity of these compounds as receptor antagonists for ERα and ERβ may be demonstrated by the assay for receptor binding activity.

### ABBREVIATIONS

Abbreviations used in the following examples and preparations include:

1,2 DCE 1,2-Dichloroethane

d Doublet

dd Double Doublet

cat. catalytic

DMAP 4-Dimethylamino Pyridine

DMSO dimethyl sulphoxide

EDC 1-(3-Dimethylaminopropyl)-3-ethylcarbodiimide Hydrochloride

EtOAc Ethyl Acetate

EtOH Ethyl Alcohol or Ethanol

Et₂O Ethyl Ether

Et₃N Triethylamine

HOBt 1-Hydroxybenzotriazole

HPLC High Pressure Liquid Chromatography

h or hr Hour(s)

m Multiplet

KHMDS Potassium hexamethylsilazide

LDA Lithium Di-isopropylamide

MeOH Methyl Alcohol or Methanol

min Minute(s)

MS Mass Spectrometry

NCS N-Chlorosuccinimde

NMR Nuclear Magnetic Resonance

PLC Preparative thin layer chromatography

PPAA 1-Propanephosphonic Acid Cyclic Anhydride

p.s.i. pounds per square inch

q Quartet

RT (or rt) room temperature (about 20-25° C.)

s Singlet

sat. Saturated

t Triplet

TBAF Tetrabutyl Ammonium Fluoride

TLC Thin Layer Chromatography

TFA Trifluoroacetic Acid

THF Tetrahydrofuran

### Example 1

4-(2-Thiophen-2-yl-1H-indol-3-yl)-phenol

Step A

N-Methoxy-2-(4-methoxy-phenyl)-N-methyl-acetamide

To a solution of 4-methoxyphenyl acetyl chloride (19.5 grams (g), 0.106 moles (mol)) in Et₂O (200 milliliter (ml)) cooled to 0° C. under an atmosphere of Nitrogen (N₂) was added pyridine (25.2 g, 0.318 mol) and DMAP (catalytic amount). N,O dimethyl hydroxyamine hydrochloride (16.5 g, 0.170 mol) was added in approximately 2 g portions over a 15 minute period. The mixture was stirred at 0° C. for 2 hours and then at room temperature overnight. The mixture was diluted with EtOAc (250 ml) and washed with 1N HCl (2×100 milliliter (ml)), water (1×100 ml) and sat. NaHCO₃ (2×100 ml). The acid washes were combined and back extracted with EtOAc (1×100 ml). All the organic layers were combined, dried (MgSO₄), filtered and concentrated in vacuo. The residue was purified by flash chromatography (SiO₂ 10% EtOAc/hexanes) to give the desired product (5.4 g, 25.8 mmol). MS (M+1) 210.

Step B

2-(4-Methoxy-phenyl)-1-thiophen-2-yl-ethanone

To a solution of thiophene (0.168 g, 2.0 mmol) in THF (3 ml) cooled to −78° C. under an atmosphere of N₂ was added n-BuLi as a 2.5M solution in hexanes (0.75 ml, 1.875 mmol). The reaction was stirred at −78° C. for 15 minutes. N-methoxy-2-(4-methoxy-phenyl)-N-methyl-acetamide (0.418 g, 2.0 mmol) was then added as a solution in THF (1 ml) to the reaction mixture. Stirring was continued at −78° C. for 30 minutes, then the cooling bath was removed and the reaction allowed to warm to room temperature. The reaction was quenched with saturated. NH₄Cl (5 ml0, diluted with water (10 ml) and extracted with EtOAc (2×30 ml). The combined organic layers were washed with brine (1×20 ml), dried (MgSO₄), filtered and concentrated in vacuo. The residue was purified by flash chromatography (SiO₂, 3% EtOAc/hexane) to give the desired product (0.128 g, 0.552 mmol). MS (M+1)⁺ 233; ¹H NMR (CDCl₃) δ_{H} 7.75 (1H, m), 7.61 (1H, m), 7.21 (2H, m), 7.11 (1H, m), 6.87 (2H, m), 4.12 (2H, s) and 3.77 (3H, s).

Step C

N-[2-(4-Methoxy-phenyl)-1-thiophen-2-yl-ethylidene]-N′-phenyl-hydrazine

A solution of 2-(4-methoxy-phenyl)-1-thiophen-2-yl-ethanone (0.122 g, 0.525 mmol), phenylhydrazine hydrochloride (0.091 g, 0.63 mmol) and Et₃N (0.090 ml, 0.63 mmol) in toluene (10 ml) was refluxed for 72 hours. The reaction mixture was diluted with EtOAc (20 ml) and washed with water (2×10 ml) and brine (1×10 ml), dried (MgSO4), filtered and concentrated in vacuo. The residue was purified by flash chromatography (3% EtOAc/hexanes to 10% EtOAc/hexanes) to give the desired product (0.080 g, 0.248 mmol). MS (M+1)⁺ 323.

Step D

3-(4-Methoxy-phenyl)-2-thiophen-2-yl-1H-indole

To a solution of N-[2-(4-methoxy-phenyl)-1-thiophen-2-yl-ethylidene]-N′-phenyl-hydrazine (0.078 g, 0.242 mmol) in CH₂Cl₂ (2 ml) was added PCl₃ as a 2.0M solution in CH₂Cl₂ (0.242 ml, 0.484 mmol). The reaction was stirred at room temperature overnight, quenched with saturated NaHCO₃, (2 ml) and extracted with EtOAc (3×10 ml). The combined extracts were washed with sat. NaHCO₃ (1×5 ml) and brine (1×5 ml), dried (MgSO₄), filtered and concentrated in vacuo. The residue was re-subjected to the same procedure again and after flash chromatography (SiO₂, 50% CH₂Cl₂/hexanes) gave the desired product (0.039 g, 0.128 mmol). MS (M+1)⁺ 306; ¹H NMR (CDCl₃) δ_{H} 8.18 (1H, s), 7.52 (1H, d, J 8.0 Hz), 7.39 (3H, m), 7.21 (2H, m), 7.11 (2H, m), 6.98 (3H, m) and 3.87 (3H, s).

Step E

4-(2-Thiophen-2-yl-1H-indol-3-yl)-phenol

To a solution of 3-(4-methoxy-phenyl)-2-thiophen-2-yl-1H-indole (0.033 g, 0.108 mmol) in CH₂Cl₂ (1 ml) cooled to −78° C. under an atmosphere of N₂ was added BBr₃ as a 1.0M solution in CH₂Cl₂. The reaction was stirred overnight slowly warming to room temperature. The reaction was quenched by the addition of MeOH (0.5 ml) and sat. NaHCO₃ was added until the pH>7. The mixture was extracted with EtOAc (3×10 ml). The combined organics were washed with sat. NaHCO₃ (1×10 ml) and brine (1×10 ml), dried (MgSO₄), filtered and concentrated in vacuo. The residue was purified by flash chromatography (SiO₂, 25% EtOAc/hexanes) to give the desired product (0.028 g, 95.8 μmol). MS (M+1)⁺ 293; ¹H NMR (CDCl₃) δ_{H} 8.17 (1H, s), 7.50 (1H, d J 8.0 Hz), 7.36 (3H, m), 7.22 (2H, m), 7.08 (2H, m), 6.98 (1H, m) and 6.88 (2H, m).

### Example 2

4-(2-Phenyl-1H-indol-3-yl)-phenol

Step A

To a solution of 2-(4-methoxy-phenyl)-1-phenyl-ethanone (0.299 g, 1.32 mmol) and phenylhydrazine hydrochloride (0.229 g, 1.58 mmol) in EtOH was added HCl as a 4.0M solution in 1,4 dioxane (0.1 ml). The reaction was heated under reflux overnight. Upon cooling to room temperature, the mixture was diluted with CH₂Cl₂ (50 ml) and washed with water (2×20 ml), sat. NaHCO₃ (2×20 ml) and brine (1×2 ml), dried (MgSO₄), filtered and concentrated in vacuo. The residue was purified by flash chromatography (SiO₂, 3% EtOAc/hexanes) to give 3-(4-methoxy-phenyl)-2-phenyl-1H-indole (0.152 g, 0.508 mmol). MS (M+1) 300.

Step B

4-(2-Phenyl-1H-indol-3-yl)-phenol

To a suspension of 3-(4-methoxy-phenyl)-2-phenyl-1H-indole (0.050 g, 0.167 mmol) in CH₂Cl₂ at −78° C. under an atmosphere of N₂ was added BBr₃ as a 1.0M solution in CH₂Cl₂. The reaction was stirred overnight, slowly warming to room temperature. MeOH (1.0 ml) was added to quench the reaction and sat. NaHCO₃ was added adjusting the pH>7. The mixture was diluted with water (10 ml) and extracted with CH₂Cl₂ (2×15 ml). The combined organics were washed with brine (1×20 ml), dried (MgSO₄), filtered and concentrated in vacuo. The residue was purified by flash chromatography (SiO₂, CH₂Cl₂) to give the desired product (0.038 g, 0.133 mmol). MS (M+1) 286.

### Example 3

4-[2-(4-Methoxy-phenyl)-1H-indol-3-yl]-phenol and

### (ii) Example 4

2,3-Bis-(4-hydroxy-phenyl)-1H-indole

2,3-Bis-(4-methoxy-phenyl)-1H-indole can be prepared according to the procedure of Letcher, Roy M.; Wai, John S. M. J. Chem. Res. Miniprint; 2; 1986; 0514-0536.

To a solution of 2,3-Bis-(4-methoxy-phenyl)-1H-indole (0.085 g, 0.258 mmol) in CH₂Cl₂ (3 ml) cooled to −78° C. under an atmosphere of N₂ was added BBr₃ as a 1.0M solution in CH₂Cl₂ (0.78 ml, 0.78 mmol). The reaction was stirred overnight slowly warming to room temperature upon which it was quenched with MeOH (5 ml). The mixture was diluted with CH₂Cl₂ (20 ml) and washed with sat. NaHCO₃ (1×2 ml), back extracted with CH₂Cl₂ (1×50 ml). The combined organic layer was dried (MgSO₄), filtered and concentrated in vacuo. The residue was purified by preparative flash chromatography (SiO₂, 1:2 EtOAc:hexanes) to give;

(i) Example 3: 4-[2-(4-Methoxy-phenyl)-1H-indol-3-yl]-phenol (0.025 g, 79.4 μmol). MS (M+1) 316.

and (ii) Example 4 : 2,3-Bis-(4-hydroxy-phenyl)-1H-indole (0.050 g, 0.166 mmol). MS (M+1) 302. ¹H NMR (CD₃OD) δ_{H} 7.42 (1H, m), 7.33 (1H, m), 7.27 (2H, d, J 8.5 Hz), 7.16 (2H, d, J 8.5 Hz), 7.05 (1H, m), 6.94 (1H, m), 6.78 (2H, d, J 8.5 Hz) and 6.68 (2H, d, J 8.5 Hz).

### Example 5

4-(3-Phenyl-1H-indol-2-yl)-phenol

4-(3-Phenyl-1H-indol-2-yl)-phenol was prepared in analogous manner to that as described for example 2 except 1-(4-hydroxy-phenyl)-2-phenyl-ethanone was used. MS (M+1) 286.

### Example 6

5-Fluoro-2,3-diphenyl-1H-indole

5-Fluoro-2,3-diphenyl-1H-indole was prepared in analogous manner to that as described for example 2 except that (4-fluoro-phenyl) hydrazine hydrochloride and 1,2 diphenyl-ethanone were used. MS (M+1) 288.

### Example 7

2,3-Bis-(4-hydroxy-phenyl)4-chloro-1H-indole

4-Chloro-2,3-bis-(4-hydroxy-phenyl)-1H-indole was prepared in analogous manner to that as described for example 2 except that (3-chloro-phenyl)-hydrazine hydrochloride and 1,2 bis-(4-methoxy-phenyl)-ethanone were used. MS (M+1) 320.

### Example 8

2,3-Bis-(4-hydroxy-phenyl)-5-bromo-1H-indole

5-Bromo-2,3-bis-(4-hydroxy-phenyl)-1H-indole was prepared in analogous manner to that as described for example 2 except (4-bromo-phenyl) hydrazine hydrochloride and 1,2 bis-(4-methoxy-phenyl)-ethanone were used. MS (M+1) 380.

### Example 9

2,3-Diphenyl-1H-indol-6-ol

2,3-Diphenyl-1H-indol-5-ol was prepared in analogous manner to that as described for example 2 except that (3-methoxy-phenyl)-hydrazine hydrochloride and 1,2 diphenyl-ethanone were used. MS (M+1) 286.

### Example 10

4-(7-Chloro-2-phenyl-1H-indol-3-yl)-phenol

4-(7-Chloro-2-phenyl-1H-indol-3-yl)-phenol was prepared in analogous manner to that as described for example 2 except that (2-chloro-phenyl)-hydrazine hydrochloride was used. MS (M+1) 320.

### Example 11

4-{2-[4-(2-Pyrrolidin-1-yl-ethoxy)-phenyl]-1H-indol-3-yl}-phenol 1-(2-phenoxy-ethyl)-pyrrolidine

To a solution of 1-[2-(4-bromo-phenoxy)-ethyl]-pyrrolidine (5.41 g, 20.0 mmol) in THF (60 ml) cooled to −78° C. under an atmosphere of N₂ was added n-BuLi as a 2.5M solution in hexanes (9.0 ml, 22.5 mmol). The reaction was stirred at −78° C. for 45 minutes then sat. NH₄Cl solution (20 ml) was added. The mixture was allowed to warm to room temperature. The mixture was diluted with water (100 ml) and extracted with EtOAc (3×100 ml). The combined organics were washed with water (1×100) and brine (1×100 ml), dried (MgSO₄), filtered and concentrated in vacuo. MS (M+1) 192.

2-(4-Methoxy-phenyl)-1-[4-(2-pyrrolidin-1-yl-ethoxy)-phenyl]-ethanone

To a solution of 1-(2-phenoxy-ethyl)-pyrrolidine (2.73 g, 14.3 mmol) and phenacetyl chloride (3.95 g, 21.4 mmol) in CH₂Cl₂ (40 ml) cooled to 0° C. was added AlCl₃ (3.81 g, 28.6 mmol) over a period of 10 minutes. The reaction mixture was stirred at 0° C. slowly warming to room temperature over a period of 48 hours. The reaction was poured onto ice and extracted with EtOAc (3×400 ml). The combined EtOAc extracts were washed with brine (1×300 ml), dried (MgSO₄), filtered and concentrated in vacuo. The material was taken directly on. MS (M+1) 340.

4-{2-[4-(2-Pyrrolidin-1-yl-ethoxy)-phenyl]-1H-indol-3-yl}-phenol

Using 2-(4-methoxy-phenyl)-1-[4-(2-pyrrolidin-1-yl-ethoxy)-phenyl]-ethanone, 4-{2-[4-(2-pyrrolidin-1-yl-ethoxy)-phenyl]-1H-indol-3-yl}-phenol was prepared in an analogous manner to that as described in example 2. MS(M+1) 399.

### Example 12

4-(1-Methyl-2-phenyl-1H-indol-3-yl)-phenol

Step A 3-(4-Methoxy-phenyl)-1-methyl-2-phenyl-1H-indole

To a solution of 3-(4-methoxy-phenyl)-2-phenyl-1H-indole (0.55 mg, 0.184 mmol), in Et₂O (2 ml) was added NaH (10 mg of 60% dispersion on oil, 0.25 mmol). The reaction was stirred at room temperature until the NaH had dissolved. Methyl iodide (0.114 g, 0.803 mmol) was added and stirring was continued overnight at room temperature. The reaction was quenched with water (2 ml) and extracted with EtOAc (2×5 ml). The combined organics were dried (Na₂SO₄), filtered and concentrated in vacuo. The residue was purified by preparative TLC (SiO₂, 25% EtOAc/hexanes) to give the desired product (0.075 g, 86.3 μmol). MS (M+1) 314.

Step B 4-(1-Methyl-2-phenyl-1H-indol-3-yl)-phenol

To a solution of 3-(4-methoxy-phenyl)-1-methyl-2-phenyl-1H-indole (0.022 g, 70.3 μmol) in CH₂Cl₂ (1 ml) cooled to −78° C. under an atmosphere of N₂ was added BBr₃ as a 1.0M solution in CH₂Cl₂ (0.28 ml, 0.28 mmol). The reaction was stirred overnight slowly warming to room temperature upon which it was quenched with MeOH (0.5 ml). The pH of the solution was adjusted to pH=7 by the addition of sat. NaHCO₃. The mixture was extracted with EtOAc (3×4 ml). The combined organics were washed with brine (1×2 ml), dried (MgSO₄), filtered and concentrated in vacuo. The residue was purified by preparative TLC (SiO₂, 30% EtOAc/hexanes) to give the desired product (0.015 g, 50.1 μmol). MS (M+1) 300.

### Example 13

4-(1-Ethyl-2-phenyl-1H-indol-3-yl)-phenol

4-(1-Ethyl-2-phenyl-1H-indol-3-yl)-phenol was prepared in a manner analogous to that as described example 12 except ethyl iodide was used.

### Example 14

5-Chloro-2,3-di-o-tolyl-1H-indole

was prepared in a manner analogous to that as described example 2 except that (4-chloro-phenyl) hydrazine hydrochloride and 1,2 di-o-tolyl-ethanone were used and step B was omitted.

### Example 15

2,3-Bis-(4-methoxy-phenyl)-1H-indole

was prepared in a manner analogous to that as described by Letcher, Roy M.; Wai, John S. M. J. Chem. Res. Miniprint; 2; 1986; 0514-0536. except BBr₃ was used to effect removal of the methoxy groups.

### Example 16

5-Chloro-2,3-diphenyl-1H-indole

was prepared in a manner analogous to that as described example 2 except that (4-chloro-phenyl) hydrazine hydrochloride and 1,2 diphenyl ethanone were used and step B omitted.

### Example 17

2-(4-Methoxy-phenyl)-3-phenyl-1H-indole

was prepared in a manner analogous to that as described example 2 except that 1-(4-methoxy-phenyl)-2-phenyl-ethanone was used.

### Example 18

2,3-Bis-(4-fluoro-phenyl)-1H-indole

was prepared in a manner analogous to that as described example 2 except that 1,2 bis-(4-fluoro-phenyl) ethanone and step B was omitted.

### Example 19

4-[5-Chloro-3-(4-methoxy-phenyl)-1H-indol-2-yl-phenol

was prepared in a manner analogous to that as described example 2 except that (4-chloro-phenyl) hydrazine hydrochloride and 1,2 bis-(4-methoxy-phenyl)-ethanone were used.

### Example 20

2,3-Bis-(4-hydroxy-phenyl)-5-chloro-1H-indole

was prepared in a manner analogous to that as described example 2 except that (4-chloro-phenyl) hydrazine hydrochloride and 1,2 bis-(4-methoxy-phenyl)-ethanone were used.

### Example 21

2,3-Bis-(4-hydroxy-phenyl)-7-chloro-1H-indole

was prepared in a manner analogous to that as described example 2 except that (2-chloro-phenyl)-hydrazine hydrochloride and 1,2 bis-(4-methoxy-phenyl)-ethanone were used.

### Example 22

2,3-Bis-(4-hydroxy-phenyl)-6-chloro-1H-indole

was prepared in a manner analogous to that as described example 2 except that (3-chloro-phenyl)-hydrazine hydrochloride and 1,2 bis-(4-methoxy-phenyl)-ethanone was used.

### Example 23

4-[5-Bromo-2-(4-methoxy-phenyl)-1H-indol-3-yl]-phenol

was prepared in a manner analogous to that as described example 2 except that (4-bromo-phenyl) hydrazine hydrochloride and 1,2 bis-(4-methoxy-phenyl)-ethanone was used.

### Example 24

2,3-Diphenyl-1H-indol-4-ol

was prepared in a manner analogous to that as described example 2 except that (3-methoxy-phenyl) hydrazine hydrochloride and 1,2 diphenyl ethanone were used.

### Example 25

2,3-Bis-(4-hydroxy-phenyl)-1-methyl-indole

was prepared in a manner analogous to that described in example 12 except that 2,3-bis-(4-methoxy-phenyl)-1H-indole was used.

1. A compound of formula (I) 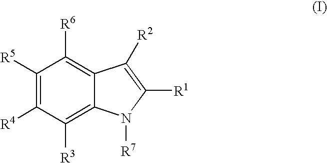
or the pharmaceutically acceptable salts thereof; wherein:
R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl;
wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:
halogen; (C₁-C₆)alkyl; (C₃-C₈)cycloalkyl; (C₄-C₈)cycloalkenyl; (C₁-C₆)alkoxy; hydroxy; R¹²CO₂, R¹²R¹³NCO, R¹²R¹³N; (C₁-C₆)alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)alkoxy; R¹²R¹³N(C₁-C₆alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:
halogen, (C₁-C₆)alkyl; (C₃-C₈)cycloalkyl; (C₄-C₈)cycloalkenyl; (C₁-C₆)alkoxy; hydroxy; R¹²CO₂; R¹²R¹³NCO; R¹²R¹³N;(C₁-C₆)alkylcarbonyl; —CHO; cyano; thio; R¹² SO₂(C₁-C₆)alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆)alkoxy; R¹²CO(C₁-C₆)alkoxy; R¹²R¹³N SO₂(C₁-C₆)alkyl; and R¹²R¹³N SO₂(C₁-C₆) alkoxy;
wherein:
R¹² and R¹³ are each independently selected from the group consisting of hydrogen; halogen; (C₁-C₇)alkyl; (C₃-C₈)cycloalkyl; (C₄-C₈)cycloalkenyl;(C₆-C₁₀) aryl; (C₂-C₁₀)alkenyl; (C₂-C₁₀)alkynyl; (C₂-C₄)heteroaryl; (C₁-C₆)alkylaryl; (C₁-C₆) alkyl(C₂-C₆)heteroaryl; (C₂-C₆)alkoxyaryl ; (C₂-C₆) alkoxy(C₂-C₆)heteroaryl; or R¹² and R¹³ taken together form a three to eight membered heterocyclic ring having 1 to 3 heteroatoms; n is from 0 to 5; and x is 1 or 2;
or R¹ and R² are each independently a group of the formula: 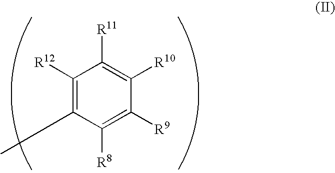
wherein R⁸, R⁹, R¹¹ and R¹² are each independently hydrogen; hydroxy; (C₁-C₆)alkyl; (C₁-C₆)alkoxy; or halogen;
R¹⁰ is hydrogen; hydroxy; (C₁-C₆) alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy;
R³, R⁴, R⁵ and R⁶ are each independently hydrogen, hydroxy; (C₁-C₆)alkyl; (C₁-C₆)alkoxy; or halogen; and
R⁷ is H or (C₁-C₃)alkyl;
with the proviso that at least one of R¹ or R² must be the group of formula (II) and with the proviso that when R¹ and R² are each independently the group of Formula II, wherein each R¹⁰ is hydrogen or hydroxy, then at least one of R³, R⁴, R⁵or R⁶ must be other than hydrogen, hydroxy or (C₁-C₆)alkoxy.

2. A compound according to claim 1, wherein R¹ is phenyl or (C₂-C₆) heteroaryl.

3. A compound according to claim 2, wherein the (C₂-C₆) heteroaryl is thienyl; furyl; pyrrolyl; isoxazolyl; isothiazoyl or thiodiazolyl.

4. A compound according to claim 1, wherein R² is a group of formula (II).

5. A compound according to claim 4, wherein R⁸, R⁹, R¹¹ and R¹² are hydrogen and R¹⁰ is hydroxy or (C₁-C₆)alkoxy.

6. A compound according to claim 1, wherein R³, R⁴, R⁵ and R⁶ are hydrogen.

7. A compound according to claim 1, wherein R¹ is phenyl or (C₂-C₆)heteroaryl; R² is a group of formula (II); and R³, R⁴, R⁵ and R⁶ are hydrogen.

8. A compound according to claim 7, wherein (C₂-C₆)heteroaryl is thienyl; furyl; pyrrolyl; isoxazolyl; isothiazoyl or thiodiazolyl; R⁸, R⁹, R¹¹ and R¹² are hydrogen; and R¹⁰ is hydroxy or (C₁-C₆)alkoxy.

9. A compound according to claim 1, wherein the compound of Formula (I) is selected from the group consisting of:
2,3-Bis-(4-methoxy-phenyl)-1H-indole;
5-Chloro-2,3-diphenyl-1H-indole;
5-Chloro-2,3-bis-(2-chloro-phenyl)-1H-indole;
4-[2-(4-Methoxy-phenyl)-1H-indol-3-yl]-phenol;
5-Fluoro-2,3-diphenyl-1H-indole;
2-(4-Methoxy-phenyl)-3-phenyl-1H-indole;
2,3-Bis-(4-fluoro-phenyl)-1H-indole;
4-[5-Chloro-3-(4-methoxy-phenyl)-1H-indol-2-yl]-phenol;
2,3-Bis-(4-hydroxy-phenyl)-5-chloro-1H-indole;
2,3-Bis-(4-hydroxy-phenyl)-7-chloro-1H-indole;
2,3-Bis-(4-hydroxy-phenyl)-4-chloro-1H-indole;
2,3-Bis-(4-hydroxy-phenyl)-6-chloro-1H-indole;
4-[5-Bromo-2-(4-methoxy-phenyl)-1H-indol-3-yl]-phenol;
2,3-Bis-(4-hydroxy-phenyl)-6-chloro-1H-indole;
2,3-Diphenyl-1H-indol-4-ol;
4-(7-Chloro-2-phenyl-1H-indol-3-yl)-phenol;
2,3-Bis-(4-hydroxy-phenyl)-1-methyl-indole;
4-(2-Thiophen-2-yl-1H-indol-3-yl)-phenol;
4-{2-[4-(2-Pyrrolidin-1-yl-ethoxy)-phenyl]-1H-indol-3-yl}-phenol.
4-[2-(1-Methyl-1H-pyrrol-2-yl)-1H-indol-3-yl]-phenol;
4-[2-(3-Methyl-isoxazyl-4-yl)-1H-indol-3-yl]-phenol;
4-[2-(5-Methyl-isoxazyl-4-yl)-1H-indol-3-yl]-phenol;
4-[2-(3,5-Dimethyl-isoxazyl-4-yl)-1H-indol-3-yl]-phenol;
4-[3-(4-Hydroxy-phenyl)-1H-indol-2-yl]-benzoic acid methyl ester;
4-[3-(4-Hydroxy-phenyl)-1H-indol-2-yl]-benzoic acid ethyl ester;
4-[3-(4-Hydroxy-phenyl)-1H-indol-2-yl]-benzoic acid isopropylester;
4-(2-Isothiazol-5-yl-1H-indol-3-yl)-phenol;
4-[2-(4-Methyl-isothiazol-5-yl)-1H-indol-3-yl]-phenol;
4-(2-Cyclopropyl-1H -indol-3-yl)-phenol;
4-[2-(3-Ethyl-isoxazyl-4-yl)-1H-indol-3-yl]-phenol;
4-[2-(5-Methyl-furan-3-yl)-1H-indol-3-yl]-phenol; and
4-(2-Furan-3-yl-1H-indol-3-yl)-phenol.

10. A pharmaceutical composition for antagonizing or agonizing the estrogen receptor in a mammal comprising an estrogen receptor antagonizing or agonizing effective amount of a compound of formula (I) according to claim 1, or a pharmaceutically accepted salt thereof, and a pharmaceutically acceptable carrier.

11. A pharmaceutical composition for selectively antagonizing or agonizing the ERβ estrogen receptor in a mammal comprising an ERβ estrogen receptor antagonizing or agonizing effective amount of a compound of formula (I) or the pharmaceutically acceptable salts thereof 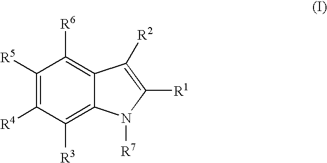
wherein:
R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl;
wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:
halogen; (C₁-C₆)alkyl; (C₃-C₈)cycloalkyl; (C₄-C₈)cycloalkenyl; (C₁-C₆)alkoxy; hydroxy; R¹²CO₂; R¹²R¹³NCO; R¹²R¹³N; (C₁-C₆)alkylcarbonyl; —CHO; cyano; thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆)alkoxy; R¹²R¹³N(C₁-C₆)alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:
halogen, (C₁-C₆)alkyl; (C₃-C₈)cycloalkyl; (C₄-C₈)cycloalkenyl; (C₁-C₆)alkoxy; hydroxy; R¹²CO₂; R¹²R¹³NCO; R¹²R¹³N;(C₁-C₆)alkylcarbonyl; —CHO; cyano; thio; R¹² SO₂ (C₁-C₆) alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆) alkoxy; R¹²CO(C₁-C₆)alkoxy; R¹²R¹³N SO₂(C₁-C₆)alkyl; and R¹²R¹³N SO₂(C₁-C₆)alkoxy
wherein:
R¹² and R¹³ are each independently selected from the group consisting of hydrogen; halogen; (C₁-C₇)alkyl; (C₃-C₈)cycloalkyl; (C₄-C₈)cycloalkenyl; (C₆-C₁₀) aryl; (C₂-C₁₀)alkenyl, (C₂-C₁₀)alkynyl; (C₂-C₄)heteroaryl; (C₁-C₆)alkylaryl; (C₁-C₆)alkyl (C₂-C₆)heteroaryl; (C₂-C₆) alkoxyaryl; (C₂-C₆)alkoxy(C₂-C₆)heteroaryl; or R¹² and R¹³ taken together form a three to eight membered heterocyclic ring having up to 3 heteroatoms; n is from 0 to 5; and x is 1 or 2;
or R¹ and R² are each independently a group of the formula: 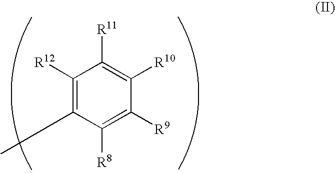
wherein R⁸, R⁹, R¹¹ and R¹²are each independently hydrogen; hydroxy; (C₁-C₆)alkyl; (C₁-C₆)alkoxy; or halogen;
R¹⁰ is hydrogen; hydroxy; (C₁-C₆)alkoxy; (C₁-C₆)alkoxycarbonyloxy; (C₁-C₆)alkylcarbonyloxy; (C₃-C₈)cycloalkoxy; (C₄-C₈) cycloalkenyloxy; or (C₆-C₁₂) aryloxy;
R³, R⁴, R⁵ and R⁶ are each independently hydrogen; hydroxy; (C₁-C₆)alkyl; (C₁-C₆)alkoxy; or halogen; and
R⁷ is H or (C₁-C₃)alkyl;
with the proviso that at least one of R¹ or R² must be the group of formula (II); and a pharmaceutically acceptable carrier.

12. A pharmaceutical composition comprising an agent selected from the group consisting of an anabolic agent; a growth hormone; a growth hormone secretagogue; a prostaglandin agonist/antagonist; a parathyroid hormone; sodium fluoride; and a mixture thereof; the pharmaceutical composition further comprising a compound of formula (I) according to claim 1.

13. A method of treating a condition which presents with low bone mass in a mammal comprising administering to the mammal a compound of formula (I) according to claim 1, a prodrug thereof or a pharmaceutically acceptable salt, or a diastereomeric mixture of said compound, salt or prodrug.

14. The method of claim 13 wherein the condition is osteoporosis.

15. A kit comprising: a) an amount of a compound of Formula (I) as defined in claim 1; b) an amount of a second compound an anabolic agent; a growth hormone; a growth hormone secretagogue; a prostaglandin agonist/antagonist; a parathyroid hormone; sodium fluoride; or a mixture thereof; and c) a container.

16. A method of treating a disease mediated by the estrogen receptor in a mammal, comprising administering to the mammal a therapeutically effective amount of a compound of formula (I) according to claim 1 in a pharmaceutically effective carrier.

17. The method of claim 16 wherein the disease is selected from the group consisting of perimenopausal or postmenopausal syndrome, osteoporosis, atrophy of skin or vagina, elevated serum cholesterol levels, cardiovascular disease, Alzheimer's disease, estrogen dependent cancers, including breast or uterine cancer, a prostatic disease, benign prostatic hyperplasia, prostate cancer, obesity, endometriosis, bone loss, uterine fibrosis, aortal smooth muscle cell proliferation, lack of birth control, acne, hirsutism, dysfunctional uterine bleeding, dysmenorrehea, male infertility, impotence, psychological and behavioral symptoms during menstruation, ulcerative mucositis, uterine fibroid disease, restenosis, atherosclerosis, musculoaponeurotic fibromatosis, alopecia, auto immune disease, cartilage degeneration, delayed puberty, demyelinating disease, dysmyelinating disease, hypoglycemia, lupus erythematosus, myocardial infection, ischemia, thromboembolic disorder, obsessive compulsive disorder, ovarian dysgenesis, post menopausal CNS disorder, pulmonary hypertension, reperfusion damage, resistant neoplasm, rheumatoid arthritis, seborrhea, sexual precocity, thyroiditis, Turner's syndrome, and hyperlipidemia and female sexual dysfunction.

18. A method for,selectively antagonizing or agonizing the ERβ estrogen receptor in a mammal comprising an ERβ estrogen receptor antagonizing or agonizing effective amount of a compound of formula (I) or the pharmaceutically acceptable salts thereof 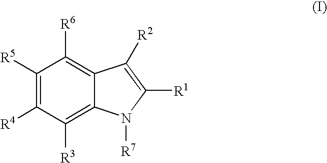
wherein:
R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl;
wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:
halogen; (C₁-C₆)alkyl; (C₃-C₈)cycloalkyl; (C₄-C₈)cycloalkenyl; (C₁-C₆)alkoxy; hydroxy; R¹²CO₂, R¹²R¹³NCO, R¹²R¹³N; (C₁-C₆) alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹²R¹³N(C₁-C₆); R¹²R¹³N(C₁-C₆alkoxy; R¹²R¹³N(C₁-C₆alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:
halogen, (C₁-C₆)alkyl; (C₃-C₈)cycloalkyl; (C₄-C₈)cycloalkenyl; (C₁-C₆)alkoxy; hydroxy; R¹² CO₂; R¹²R¹³NCO; R¹²R¹³N;(C₁-C₆)alkylcarbonyl; —CHO; cyano; thio; R¹² SO₂(C₁-C₆)alkyl; R¹²CO₂(C₁-C₆)alkyl; R¹²R¹³NCO(C₁-C₆)alkyl; R¹²CO(C₁-C₆)alkyl; R¹²SO₂(C₁-C₆)alkoxy; R¹²CO₂(C₁-C₆)alkoxy; R¹²R¹³NCO(C₁-C₆)alkoxy; R¹²CO(C₁-C₆)alkoxy; R¹²R¹³N SO₂(C₁-C₆)alkyl; and R¹²R¹³N SO₂(C₁-C₆) alkoxy;
wherein:
R¹² and R¹³ are each independently selected from the group consisting of hydrogen; halogen; (C₁-C₇)alkyl; (C₃-C₈)cycloalkyl; (C₄-C₈)cycloalkenyl; aryl; (C₂-C₁₀)alkenyl, (C₂-C₁₀)alkynyl; (C₂-C₄)heteroaryl; (C₁-C₆) alkylaryl; (C₁-C₆) alkyl(C₂-C₆) heteroaryl; (C₂-C₆) alkoxyaryl; (C₂-C₆) alkoxy(C₂-C₆)heteroaryl; or R¹² and R¹³ taken together form a three to eight membered heterocyclic ring having up to 3 heteroatoms; n is from 0 to 5; and x is 1 or 2;
or R¹ and R² are each independently a group of the formula: 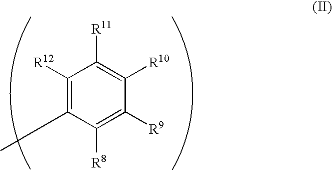
wherein R⁸, R⁹, R¹¹ and R¹² are each independently hydrogen; hydroxy; (C₁-C₆) alkyl; (C₁-C₆)alkoxy; or halogen;
R¹⁰ is hydrogen; hydroxy; (C₁-C₆)alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy;
R³, R⁴, R⁵and R⁶ are each independently hydrogen, hydroxy; (C₁-C₆) alkyl; (C₁-C₆)alkoxy; or halogen; and
R⁷ is H or (C₁-C₃)alkyl;
with the proviso that at least one of R¹ or R² must be the group of formula (II).
